(19) United States
(12) Patent Application Publication
(10) Pub. No.: US 2019/0194591 A1
(43) Pub. Date: Jun. 27, 2019

(54) SUPPORT FOR CULTURING CELLS, METHOD OF PREPARING SUPPORT, AND METHOD OF CULTURING CELLS

(71) Applicant: CHA University Industry-Academic Cooperation Foundation, Pocheon-si (KR)

(72) Inventors: Surin HONG, Seongnam-si (KR); Yong Soo CHOI, Gunpo-si (KR)

(73) Assignee: CHA University Industry-Academic Cooperation Foundation, Pocheon-si (KR)

(21) Appl. No.: 16/167,922

(22) Filed: Oct. 23, 2018

(30) Foreign Application Priority Data
Dec. 21, 2017 (KR) 10-2017-0177492

(51) Int. Cl. C12M 1/12; C12N 5/00

(52) U.S. Cl. CPC C12M 25/16; C12N 2533/90; C12N 2501/115; C12N 2533/10; C12N 2533/54; C12N 2501/2302; C12N 2501/2304; C12N 2501/2306; C12N 2501/165; C12N 2501/11; C12N 2501/125; C12N 2501/22; C12N 2501/24; C12N 2501/13; C12N 2533/52; C12N 2533/56; C12N 2533/32; C12N 5/0018

(57) ABSTRACT

Provided are a support for culturing cells, a method of preparing the support, and a method of culturing cells using the support. When the support and the methods are used, an adherence rate, a surface area, and a proliferation rate of cells may improve, and detachment of the cells may be facilitated, thereby increasing a cell recovery rate. In addition, a physiologically active substance may be slowly released, thereby reducing the cost for a cell culture process.

### CROSS-REFERENCE TO RELATED APPLICATION

This application claims the benefit of Korean Patent Application No. 10-2017-0177492, filed on Dec. 21, 2017, in the Korean Intellectual Property Office, the disclosure of which is incorporated herein in its entirety by reference.

### BACKGROUND

### 1. Field

One or more embodiments relate to a support for culturing cells including a silica nanoparticle bound to a physiologically active substance and a solid support, to which the silica nanoparticle is bound, a method of preparing the support, and a method of culturing cells using the support.

### 2. Description of the Related Art

Cell culture is the most fundamental research method in the field of biotechnology. Cell culture has been used widely to study human diseases as well as to study the functions of living organisms. The most commonly used method to date is to culture cells on solid supports made of polystyrene or glass.

Supports that have a porous structure have been developed to enable cells to be easily adhered, to secure a sufficient space between the cells so that oxygen and nutrients can be supplied by diffusion of body fluids, and to discharge waste products during cell culture (Korean Patent Publication No. 10-2014-0063964 A).

In addition, for cell culture, a medium contains various cell growth factors. However, such cell growth factors are rapidly exhausted during cell culture, and thus, the cell culture medium needs to be changed frequently.

Therefore, in order to culture a large number of cells, it is necessary to develop a porous support capable of containing effective factors for cell culture and slowly releasing the factors.

### SUMMARY

One or more embodiments include a support for culturing cells.

One or more embodiments include a method of preparing a support for culturing cells.

One or more embodiments include a method of culturing cells by using a support for culturing cells.

Additional aspects will be set forth in part in the description which follows and, in part, will be apparent from the description, or may be learned by practice of the presented embodiments.

According to one or more embodiments, a support for culturing cells may include silica nanoparticles each having an open stoma and a physiologically active substance included in the open stoma; and a solid support to which the silica nanoparticles may be bound.

The term “support for culturing cells” is also known as a scaffold, and refers to a physical support capable of in vitro culture of cells or tissues, ex vivo culture, or implantation.

The term “stoma” refers to a space in a particle. The open stoma may extend from the interior to a surface of a particle. The term “stoma” may be used interchangeably with a term “pore”.

The term “silica” refers to any compound including silicone dioxide (SiO₂). A silica nanoparticle may be a nanoparticle prepared from a silica precursor. The silica precursor may be, for example, tetramethyl orthosilicate, tetraethyl orthosilicate, and tetrapropyl orthosilicate.

The term “nanoparticle” refers to a particle of which a dimension may be in a range of about 1 nanometer (nm) to less than about 1000 nm. The nanoparticle may have a spherical shape, a rod shape, or an amorphous shape.

The silica nanoparticles may each have at least one open stoma. The silica nanoparticles may be in an open porous form.

The physiologically active substance may be a growth factor necessary for cell culture or a cytokine. The physiologically active substance may be selected from a basic fibroblast growth factor (bFGF), a brain-derived neurotrophic factor (BDNF), a vascular endothelial cell growth factor (VEGF), interleukin (IL)-4, IL-6, IL-2, an epidermal growth factor (EGF), a stem cell factor (SCF), an interferon gamma (IFNγ), and a granulocyte macrophage colony-stimulating factor (GM-CSF).

An inner part of the open stoma of each of the silica nanoparticles may be modified with a positively charged functional group, a negatively charged functional group, or a hydrophobic functional group. The functional group may be a hydrocarbon (e.g., alkane, alkene, alkyne, a benzene derivative, and a toluene derivative), a functional group containing a halogen, a functional group containing oxygen, a functional group containing nitrogen, a functional group containing sulfur, a functional group containing phosphorus, or a functional group containing boron. The positively charged functional group may be, for example, a —NH₃⁺ functional group. The negatively charged functional group may be, for example, a —COO⁻ functional group. The hydrophobic functional group may be, for example, an alkyl group.

The physiologically active substance may be bound to a functional group in an inner portion of a stoma of each of the silica nanoparticles. The binding may be an electrostatic bond, a chemical bond, a hydrophobic bond, or adsorption. The silica nanoparticles modified with a positively charged functional group may be electrostatically bound to a negatively charged physiologically active substance. Thus, the silica nanoparticles may contain the physiologically active substance. The silica nanoparticles modified with a negatively charged functional group may be electrostatically bound to a positively charged physiologically active substance. Thus, the silica nanoparticles may contain the physiologically active substance. The silica nanoparticles modified with a hydrophobic functional group may be hydrophobically bound to a hydrophobic charged physiologically active substance. Thus, the silica nanoparticles may contain the physiologically active substance.

The silica nanoparticles may be pH-sensitive particles, temperature-sensitive particles, or a combination thereof. By changing pH, temperature, or a combination thereof, a drug contained in the silica nanoparticles may be released. For example, the silica nanoparticles may contain a drug in an acidic condition (pH of about 2.0), and may release the drug in a neutral condition (pH of about 7.0). For example, the silica nanoparticles may contain a drug at room temperature and release the drug at body temperature. Thus, the silica nanoparticles may control the containing and releasing of a drug by controlling the pH, temperature, or a combination thereof.

The physiologically active substance may be slowly released from the silica nanoparticles. Since the physiologically active substance is slowly released from the silica nanoparticles, the physiologically active substance may not be exhausted quickly. Due to the slow release of the physiologically active substance, a replacement cycle of a cell culture medium may be prolonged, and the cost for a cell culture process may be reduced.

The silica nanoparticles may be based on cetylpyridinium bromide (CPB), cetyltrimethylammonium bromide (CTAB), or a combination thereof. The surfactant constituting a structure of the silica nanoparticles may be CPB, CTAB, or a combination thereof.

A diameter of each of the silica nanoparticles may be in a range of about 50 nm to about 3,000 nm, about 100 nm to about 2,000 nm, about 150 nm to about 1,000 nm, about 200 nm to about 900 nm, about 300 nm to about 900 nm, about 300 nm to about 800 nm, about 300 nm to about 700 nm, about 300 nm to about 600 nm, about 300 nm to about 550 nm, about 400 nm to about 900 nm, about 500 nm to about 900 nm, about 600 nm to about 900 nm, about 700 nm to about 900 nm, or about 750 nm to about 900 nm.

An average diameter of each of the silica nanoparticles may be in a range of about 100 nm to about 2,000 nm, about 200 nm to about 1,500 nm, about 300 nm to about 1,000 nm, about 400 nm to about 900 nm, or about 450 nm to about 850 nm.

A diameter of the stoma in each of the silica nanoparticles may be in a range of about 1 nm to about 100 nm, about 3 nm to about 90 nm, about 5 nm to about 80 nm, about 7 nm to about 70 nm, about 10 nm to about 60 nm, about 12 nm to about 50 nm, about 15 nm to about 40 nm, about 15 nm to about 30 nm, about 17 nm to about 50 nm, or about 20 nm to about 50 nm.

The silica nanoparticles may be bound to a solid support. The binding may be via ionic bonding or hydrophobic interaction. For example, the silica nanoparticles may be bound to the solid support via hydrophobic interaction.

The solid support may be a bead. The bead may include polystyrene, agarose, methylcellulose, hyaluronan, or a combination thereof. The solid support may have a spherical shape, an oval shape, or an amorphous shape.

The solid support may be coated with collagen, fibrin, fibronectin, vitronectin, matrigel, gelatin, laminin, heparin, poly-lysine, extracellular matrix, or a combination thereof.

A density of the silica nanoparticles on the solid support may be in a range of about 1,000/mm² to about 500,000/mm², about 2,000/mm² to about 400,000/mm², about 3,000/mm² to about 300,000/mm², about 4,000/mm² to about 200,000/mm², about 5,000/mm² to about 100,000/mm², about 6,000/mm² to about 90,000/mm², about 7,000/mm² to about 80,000/mm², about 8,000/mm² to about 70,000/mm², about 9,000/mm² to about 60,000/mm², about 10,000/mm² to about 50,000/mm², about 15,000/mm² to about 40,000/mm², about 20,000/mm² to about 30,000/mm², or about 25,000/mm² to about 30,000/mm².

The support for culturing cells may be sterilized. The sterilization may be performed at a high temperature and a high pressure.

According to one or more embodiments, a method of preparing a support for culturing cells may include: performing, in a pressure vessel, hydrothermal treatment on a solution including a silica precursor, a polar solvent, a nonpolar solvent, an ionic surfactant, and a basic compound to prepare silica nanoparticles;

incubating the silica nanoparticles and a physiologically active substance for binding the physiologically active substance to an inner portion of a stoma of each of the silica nanoparticles; and

binding, to a solid support, the silica nanoparticles to which the physiologically active substance is bound.

The silica, nanoparticles, physiologically active substance, binding, solid support, and support for culturing cells are the same as described above.

The method may include performing, in a pressure vessel, hydrothermal treatment on a solution including a silica precursor, a polar solvent, a nonpolar solvent, an ionic surfactant, and a basic compound to prepare silica nanoparticles.

The silica precursor may be an alkoxy silane compound. The silica precursor may be, for example, tetramethyl orthosilicate, tetraethyl orthosilicate, tetrapropyl orthosilicate, or a mixture thereof.

The polar solvent may be water, alcohol, or a mixture thereof. The alcohol may be, for example, propanol, butanol, pentanol, hexanol, or a mixture thereof. The nonpolar solvent may be any suitable nonpolar solvent, and is not particularly limited provided that the nonpolar solvent may react with the polar solvent to form an emulsion. For example, the nonpolar solvent may be hexane, cyclohexane, methylcyclohexane, toluene, heptane, octane, or a mixture thereof. An amount of each of the polar solvent and the nonpolar solvent is not particularly limited provided that the amount is sufficient to form an emulsion. For example, the polar solvent and the nonpolar solvent may be used at a volumetric ratio of about 0.5:1 to about 5:1 or about 0.6:1 to about 3:1.

The ionic surfactant stabilizes an interface between the polar solvent and the nonpolar solvent to form an emulsion and spatially obstructs growth of a silica bond to thereby form a stoma magnet. The surfactant may form micelles of various structures depending on an effective area of a polar head and a length and a volume of a nonpolar tail. These factors of the surfactant may be considered to form a three-dimensional (3D) open stoma structure. The surfactant may be, for example, cetylpyridinium chloride, cetylpyridinium bromide, cetyltrimetylammonium chloride, cetyltrimetylammonium bromide, or a mixture thereof.

A basic compound may be any suitable basic compound, and is not particularly limited provided that the basic compound may act as a catalyst that promotes hydrolysis and condensation of a silica precursor. For example, the basic compound may be an ammonia aqueous solution, urea, or a mixture thereof. The basic compound may be used in about 0.1 moles to about 2 moles per about 1 mole of a silica precursor.

The hydrothermal treatment may be performed using a sealed pressure vessel, such as an autoclave, without adjustment of the pressure it generates. A temperature of the hydrothermal treatment is not particularly limited provided that the hydrolysis and condensation of a silica precursor may be carried out. For example, the hydrothermal treatment may be performed at a temperature in a range of about 50° C. to about 250° C., about 80° C. to about 200° C., or about 100° C. to about 150° C.

The method may include incubating the silica nanoparticles and a physiologically active substance for binding the physiologically active substance to an inner portion of a stoma of each of the silica nanoparticles.

The binding of the physiologically active substance to the silica nanoparticles may be performed by adding the modified silica to a solution in which the physiologically active substance is dissolved, and stirring the mixture for a given time. Here, as time for stirring increases, an amount of the physiologically active substance bound to the silica nanoparticle may also increase.

The method may include binding, to a solid support, the silica nanoparticles to which the physiologically active substance is bound.

The binding of the silica nanoparticles to a solid support may be performed by mixing the silica nanoparticles with the solid support and stirring the mixture for a given time. Here, as time for stirring increases, an amount of the silica nanoparticles bound to the solid support may also increase.

The method may further include sterilizing the solid support to which the silica nanoparticles are bound.

According to one or more embodiments, a method of culturing cells may include culturing cells in the presence of the support for culturing cells according to one or more embodiments.

The support for culturing cells is the same as described above.

The cells may be derived from cells of mice, rats, rabbits, dogs, cats, sheep, cows, horses, monkeys, chimpanzees, or humans. The cells may be selected from stem cells, nerve cells, immune cells, reproductive cells, skin cells, muscle cells, blood cells, fat cells, cartilage cells, epithelial cells, and endothelial cells.

### BRIEF DESCRIPTION OF THE DRAWINGS

These and/or other aspects will become apparent and more readily appreciated from the following description of the embodiments, taken in conjunction with the accompanying drawings in which:

FIG. 1A shows field emission scanning electron microscope (FESEM) images of cetylpyridinium bromide (CPB)-based silica nanoparticles prepared according to one or more embodiments; and FIG. 1B shows FESEM images of cetyltrimethylammonium bromide (CTAB)-based silica nanoparticles;

FIG. 2A is a schematic view of a scaffold prepared according to one or more embodiments; FIG. 2B shows FESEM images of a scaffold of Col-PS-OSN-004 and a silica nanoparticle in the scaffold (left: scaffold, right: silica nanoparticle); FIG. 2C shows FESEM images of a scaffold of Col-PS-OSN-005 and a silica nanoparticle in the scaffold (left: scaffold, right: silica nanoparticle); and FIG. 2D shows FESEM images of a scaffold of Col-PS-OSN-006 and a silica nanoparticle in the scaffold (left: scaffold, right: silica nanoparticle);

FIG. 3 shows FESEM images of the scaffolds of Col-PS-OSN-001, Col-PS-OSN-002, Col-PS-OSN-003, Col-PS-OSN-004, Col-PS-OSN-005, and Col-PS-OSN-006 before and after sterilization;

FIG. 4A is a graph of absorbance (arbitrary unit, a.u.) of a contained basic fibroblast growth factor (bFGF) versus incubation time (hours); and FIG. 4B is a graph of a release rate (percentage, %) of an accumulated bFGF versus time (hours);

FIG. 5 is a graph of cell viability (compared with a control, %) versus density of silica nanoparticles (micrograms per milliliter, μg/mL); and

FIG. 6A is a graph of the total number (×10⁶) of proliferated adipocyte-derived mesenchymal stem cells (AD-MSC) while adhered to microbeads during an incubation period (days); and FIG. 6B is a graph of glucose concentration (milligrams per milliliter, mg/mL) versus incubation time (days); and FIG. 6C shows images of cells adhered to microbeads (polystyrene beads (PS, ×40) and polystyrene-OSN beads (OSN, ×40)) according to incubation time (days).

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

Hereinafter, the present disclosure will be described in more detail with reference to Examples. However, these Examples are for illustrative purposes only, and the present disclosure is not intended to be limited by these Examples.

1. Preparation of Silica Nanoparticles

(1) Preparation of Cetylpyridinium Bromide (CPB)-Based Silica Nanoparticles

1 gram (g) of CPB (available from Sigma-Aldrich), 0.6 g of urea (available from Sigma-Aldrich), and 30 milliliters (mL) of distilled water were mixed together. Then, the mixture was stirred for about 10 minutes to thereby prepare a first solution. 2.5 g of tetraethyl orthosilicate (TEOS, available from Sigma-Aldrich), 1.5 mL of 1-pentanol (available from Samchun Chemicals), and 30 mL of cyclohexane (available from Sigma-Aldrich) were mixed together. Then, the mixture was stirred for about 10 minutes to thereby prepare a second solution.

The prepared first and second solutions were mixed together. Then, the mixture was stirred for about 30 minutes at room temperature. The stirred solution was added to a teflon container and was subjected to hydrothermal treatment in a hydrothermal reactor at a temperature of about 120° C. and at a rate of 300 revolutions per minute (rpm) for about 4 hours. Subsequently, the resultant was poured into a separation funnel and was allowed to stand overnight. In the separation funnel, the layer in the middle of the three separated layers was separated. To the separated solution, the same amount of distilled water was added. Then, the mixture was centrifuged at a rate of 12,000 rpm for 30 minutes. The supernatant was removed, and then a mixture of ethanol and acetone at a ratio of 1:1 was added to the pellet. The resultant was centrifuged at a rate of 12,000 rpm for 30 minutes. The supernatant was removed, and then the pellet was dried at room temperature. The dried particle was ground in a mortar, was calcinated at a temperature of about 550° C. for about 6 hours.

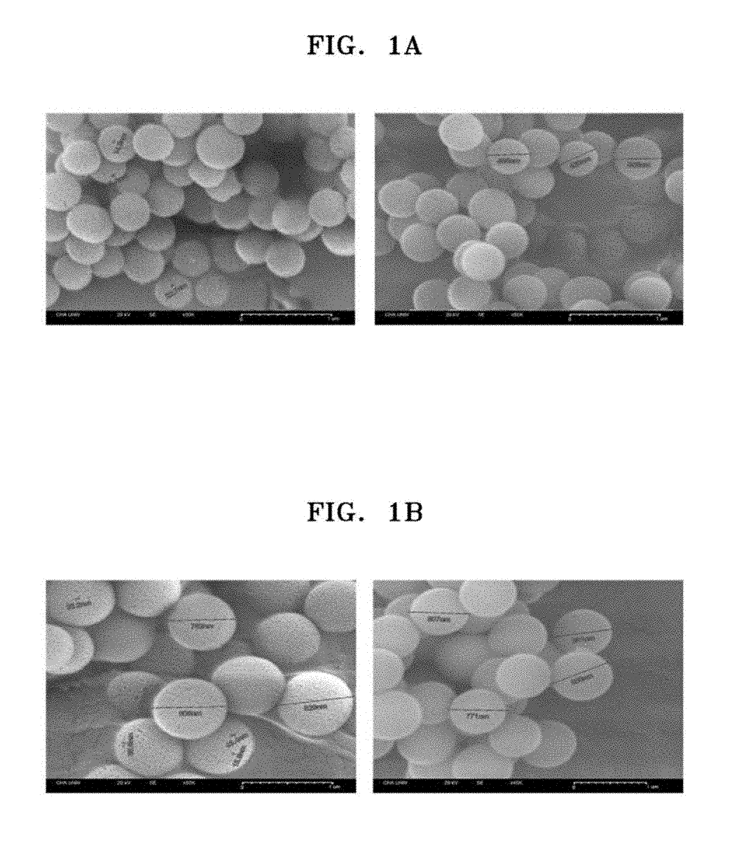

The particle size and the stoma size of the prepared silica nanoparticles were measured by using a field emission scanning electron microscope (FESEM). FIG. 1A shows a FESEM image of the nanoparticles. As shown in FIG. 1A, the size of the nanoparticles was in a range of about 300 nanometers (nm) to about 550 nm, and an average diameter of the nanoparticles was about 450 nm. Also, the size of stoma was in a range of about 15 nm to about 30 nm. The silica nanoparticles having an average diameter of about 450 nm were identified as open porous silica nanoparticles (OSN).

(2) Preparation of Cetyltrimethylammonium Bromide (CTAB)-Based Silica Nanoparticles

0.3 g of CTAB (available from Sigma-Aldrich), 0.18 g of urea (available from Sigma-Aldrich), and 30 mL of distilled water were mixed together. Then, the mixture was stirred for about 10 minutes to thereby prepare a first solution. 2.5 g of TEOS (available from Sigma-Aldrich), 1.8 mL of 1-pentanol (available from Samchun Chemicals), and 30 mL of cyclohexane (available from Sigma-Aldrich) were mixed together. Then, the mixture was stirred for about 10 minutes to thereby prepare a second solution.

Silica nanoparticles were prepared in substantially the same manner as in Example 1.(1), except that the first and second solutions prepared herein were used. The particle size and the stoma size of the prepared silica nanoparticles were measured by using a FESEM. FIG. 1B shows FESEM images of the nanoparticles. As shown in FIG. 1B, the size of the nanoparticles was in a range of about 750 nm to about 900 nm, and an average diameter of the nanoparticles was about 850 nm. Also, the size of the stoma was in a range of about 20 nm to about 50 nm. The silica nanoparticles having an average diameter of about 850 nm were identified as OSN.

2. Preparation of Slow-Releasing Scaffold and Setting Optimum Conditions

The silica nanoparticles prepared in Example 1 were modified with a functional group of —NH₃⁺.

In detail, 20 mg of N-hydroxysuccinimide (NHS) was dissolved in 8.7 mL of distilled water to prepare an NHS solution. 60 mg of 1-ethyl-3-(3-dimethylaminopropyl)-carbodiimide (EDC) was dissolved in 15.67 mL of distilled water to prepare an EDC solution. The NHS solution was mixed with the EDC solution, and the mixture was added to 0.5 g of CTAB-based OSN prepared in Example 1.(2). The reaction mixture was subjected to shaking at room temperature for about 2 hours such that the nanoparticles were coupled to carbodiimide. The reaction mixture was centrifuged to remove compounds other than OSN, NHS, and EDC, which were bound to the nanoparticles.

5 g of OSN, distilled water, and 5 g of a collagen coated polystyrene (“Col-PS”) bead (available from Sigma-Aldrich) were mixed together. The mixture was subjected to shaking for 2 hours, thereby preparing a scaffold. The mixture was filtered using a mash filter to remove aggregated silica nanoparticles. 1,000 mL of distilled water was added to the prepared scaffold, and vacuum filtration was performed thereon. The scaffold was centrifuged at a rate of 12,000 rpm for 15 minutes and washed three times, followed by drying at room temperature.

While varying the amounts of the Col-PS bead and the OSN, scaffolds were prepared in substantially the same manner. The scaffold, in which silica nanoparticles are bound to a Col-PS bead, was named “Col-PS-OSN”. The scaffolds prepared by varying the ratio of the Col-PS bead to OSN were named as in Table 1.

TABLE 1

| Col-PS bead (g) | OSN (g) | Col-PS bead:OSN | Name |
|---|---|---|---|
| 5 | 5 | 1:1 | Col-PS-OSN-001 |
| 5 | 2.5 | 1:0.5 | Col-PS-OSN-002 |
| 5 | 1.5 | 1:0.3 | Col-PS-OSN-003 |
| 5 | 0.5 | 1:0.1 | Col-PS-OSN-004 |
| 5 | 0.05 | 1:0.01 | Col-PS-OSN-005 |
| 5 | 0.005 | 1:0.001 | Col-PS-OSN-006 |

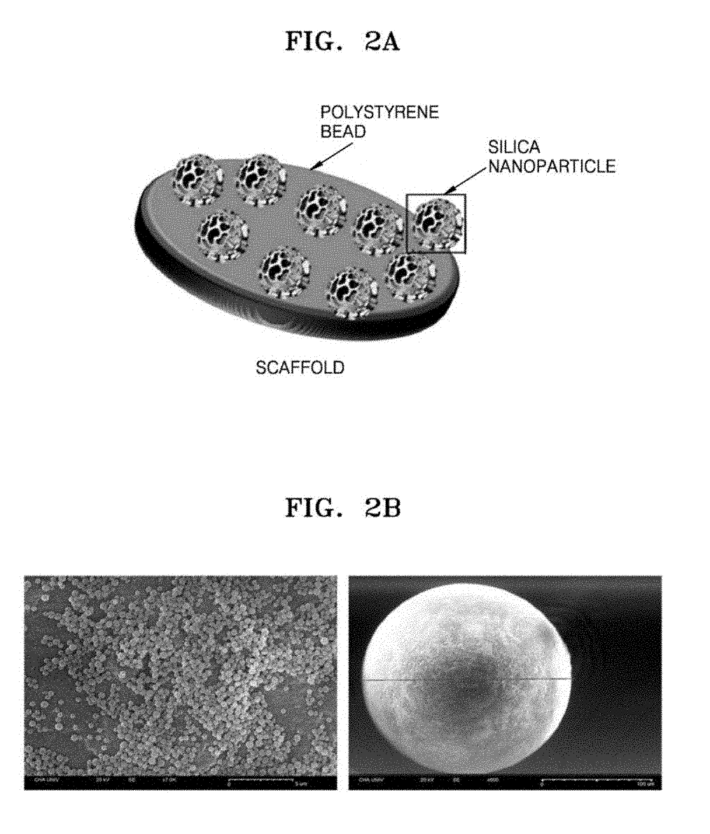

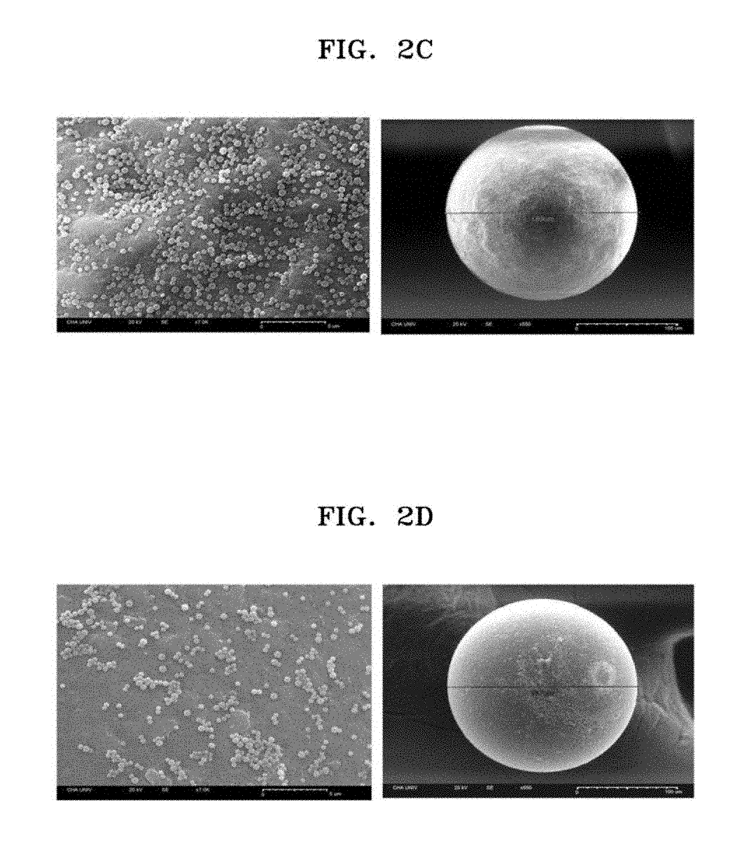

The prepared scaffold was identified by using a FESEM. FIG. 2A shows a schematic view of a scaffold. FIG. 2B shows images of a scaffold of Col-PS-OSN-004 and a silica nanoparticle bound to the scaffold. FIG. 2C shows images of a scaffold of Col-PS-OSN-005 and a silica nanoparticle bound to the scaffold. FIG. 2D shows images of a scaffold of Col-PS-OSN-006 and a silica nanoparticle bound to the scaffold. As shown in FIGS. 2A to 2C, about 35,168 silica nanoparticles were bound to the bead in Col-PS-OSN-004, about 33,438 silica nanoparticles were bound to the bead in Col-PS-OSN-005, about 19,785 silica nanoparticles were bound to the bead in Col-PS-OSN-006.

3. Sterilization of Scaffold

The scaffolds prepared in Example 2 were sterilized for the comparison of the number of silica nanoparticles bound to the bead before and after the sterilization.

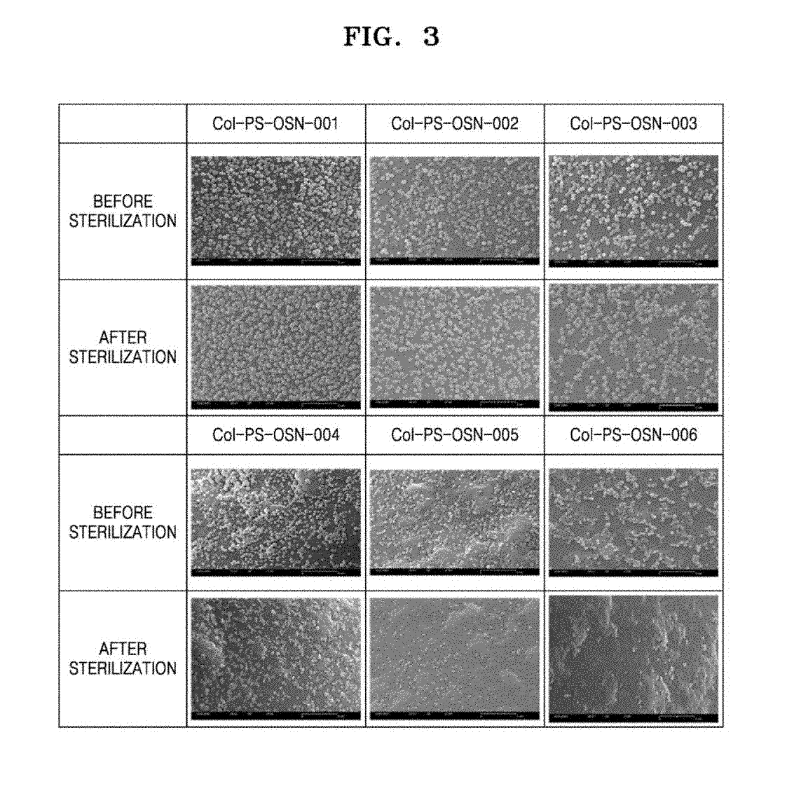

In detail, the scaffolds of Col-PS-OSN-001, Col-PS-OSN-002, Col-PS-OSN-003, Col-PS-OSN-004, Col-PS-OSN-005, and Col-PS-OSN-006 were each suspended in physiological saline solution at a concentration of 0.25 g/mL. Then, the scaffolds were moved to a sterilization container and subjected to sterilization at a temperature of 121° C. for 15 minutes in an autoclave (available from Sanyo Labo Autoclave). The scaffolds were observed by using a FESEM before and after sterilization. The images thereof are shown in FIG. 3.

The numbers of silica nanoparticles bound to the bead in each scaffold before and after sterilization are shown in Table 2.

TABLE 2

| Scaffold | Before sterilization | After sterilization | Note |
|---|---|---|---|
| Col-PS-OSN-001 | about 132,240 | about 119,952 | about 9% |
| | | | of increase |
| Col-PS-OSN-002 | about 51,080 | about 71,442 | about 23% |
| | | | of increase |
| Col-PS-OSN-003 | about 46,303 | about 46,413 | about 7% |
| | | | of increase |
| Col-PS-OSN-004 | about 35,168 | about 27,355 | about 22% |
| | | | of decrease |
| Col-PS-OSN-005 | about 33,438 | about 14,207 | about 58% |
| | | | of decrease |
| Col-PS-OSN-006 | about 19,785 | about 4,612 | about 77% |
| | | | of decrease |

4. Binding of physiologically active factors to scaffold

Col-PS-OSN-004 prepared in Example 3 (Col-PS bead:OSN=1:0.1) was modified with −NH₃⁺ to load a negatively charged drug.

In detail, about 1 g of silica nanoparticles were suspended in about 80 mL of toluene (available from Sigma-Aldrich), and about 4 mL of 3-aminopropyltrimethoxysilane (APTMS)(available from Sigma-Aldrich) was added thereto. This solution was stirred at a temperature of about 110° C. for about 24 hours, followed by washing with ethanol and centrifugation, thereby obtaining silica nanoparticles to which functional groups were bound. For the binding functional groups to be more positively charged, about 0.3 g of silica nanoparticles, to which functional groups were bound, were suspended in about 20 mL of alcohol. Then, treatment with an acidic condition using about 0.1 M of a hydrochloric acid aqueous solution was additionally performed.

Subsequently, a basic fibroblast growth factor (bFGF, available from CHA MEDITECH) was added to the solution, followed by stirring at room temperature for about 4 hours and centrifugation, thereby obtaining silica nanoparticles loading a drug.

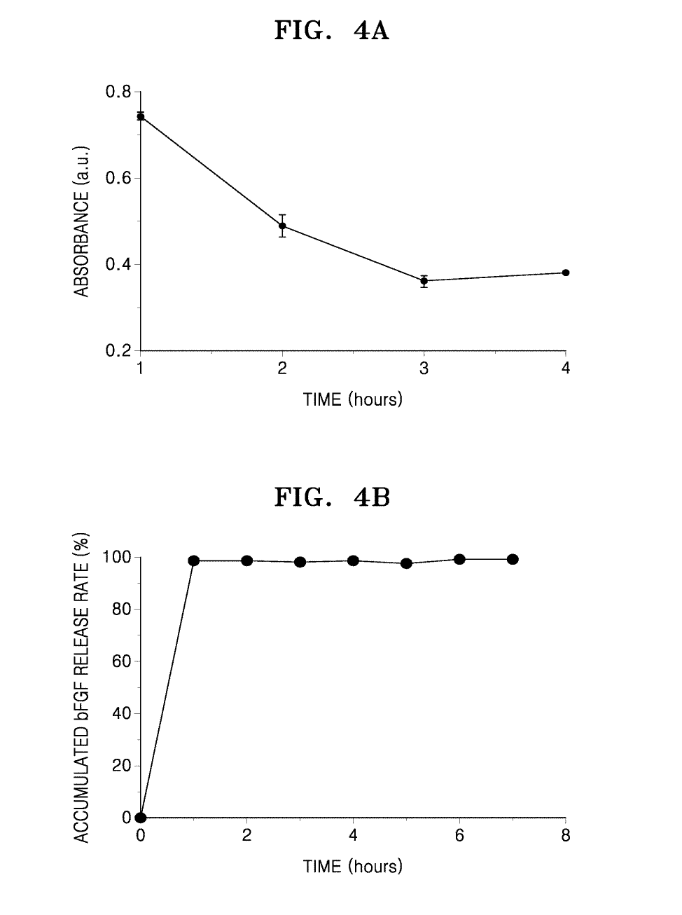

The amount of the loaded drug was calculated by observing a concentration of the drug detected by measuring UV absorbance (at a wavelength in a range of 288 nm to 290 nm) of an ethanol solution in which the drug was dissolved, before and after addition of the drug. FIG. 4A is a graph of absorbance (arbitrary unit, a.u.) versus incubation time (hours). Also, the amount (mg) of bFGF in the sample, the amount (mg) of bFGF loaded, the bFGF loading efficiency (%), and the loading efficiency of OSN per g (%) are shown in Table 3.

TABLE 3

| Amount of | | | | Loading |
|---|---|---|---|---|
| bFGF in the | Amount of | bFGF loading | Dry OSN | efficiency |
| sample | loaded bFGF | efficiency | weight | of OSN per g |
| (mg) | (mg) | (%) | (mg) | (%) |
| 0.637 | 0.3 | 47 | 6 | 5 |
| 0.654 | 0.32 | 49 | 5 | 6.44 |
| 0.626 | 0.3 | 48 | 5 | 6 |

As shown in FIG. 4A and Table 3, when bFGF was loaded by using OSN, the bFGF loading efficiency was about 48%, and the loading efficiency of OSN per g was about 6%. Accordingly, depending on the amount of reactive materials that initially react, the loading efficiency was improved.

Also, for a drug release test, about 10 mg of OSN was added to about 2 mL of a phosphate buffered saline (PBS) aqueous solution, followed by stirring while maintaining the solution at a temperature of 36.5° C. At an interval of 1 hour, about 1 mL of the sample was collected from the solution. The collected solution was centrifuged to thereby obtain a supernatant. Subsequently, UV absorbance of the supernatant was measured to calculate the drug release rate at a wavelength in a range of 288 nm to 290 nm.

Table 4 shows absorbance over time, and FIG. 4B shows drug release rate (%).

TABLE 4

| Time (hour) | Absorbance (a.u.) |
|---|---|
| 0 | 0.062 |
| 1 | 0.568 |
| 2 | 0.578 |
| 3 | 0.557 |
| 4 | 0.569 |
| 5 | 0.546 |
| 6 | 0.572 |
| 7 | 0.567 |

Referring to Table 4 and FIG. 4B, it was found that the loaded bFGF was released from OSN rapidly.

5. Effects of Scaffold in Cell Culture

(1) Test of Cytotoxicity

The cytotoxicity of the OSN, i.e., an essential component, of Col-PS-OSN-004 (Col-PS bead:OSN=1:0.1) prepared in Example 3 was tested.

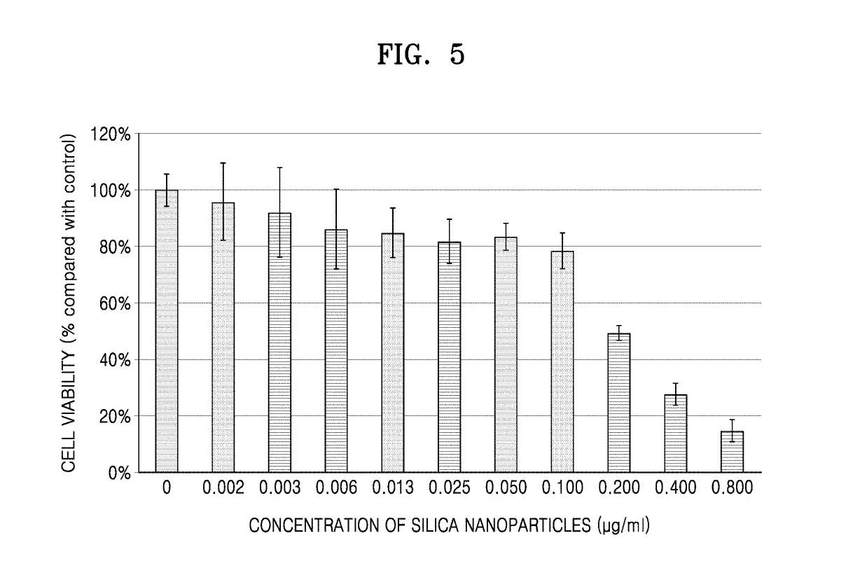

The measurement of cytotoxicity was performed by the WST-1 (EZ cytox available from Dogenbio) measurement method. The results thereof are shown in FIG. 5. For cytotoxicity measurement, 2,000 adipocyte-derived mesenchymal stem cells (AD-MSC) were inoculated to each well of a 96-well plate (available from SPL). Each silica nanoparticle was added thereto at a concentration in a range of 0.002 μg/mL to 0.8 μg/mL. Then, the cell culture solution was adjusted to a volume of 200 μL. This 96-well plate was subjected to incubation in an incubator at a temperature of 37° C. and 5% CO₂ for 3 days. In order to measure the cell proliferation rate, 20 μL of WST-1 reagent, which corresponds to 10% of the total culture solution, was added to each well. Then, a reaction was performed in an incubator at a temperature of 37° C. and 5% CO₂ for 2 hours. Once the reaction was complete, absorbance (at 450 nm) was measured by using a microplate reader (available from BioTek instruments). The cell viability percentage was calculated using the measured absorbance value, based on the value of a 96-well plate to which silica nanoparticles were not added. In FIG. 5, the cell viability is shown on the Y-axis and the concentration of silica nanoparticles is shown on the X-axis.

As shown in FIG. 5, at a concentration of OSN of about 200 μg/mL or higher, cell growth was found to be inhibited (<50%)

(2) Cell Culture by Using Scaffolds

PS-OSN prepared in Example 3 and Col-PS microbeads (available from PALL Cooperation), i.e., a control, were used for the comparison of cell proliferation effects regarding mesenchymal stem cells derived from human adipose tissue.

First, AD-MSCs (available from Cha Bundang Medical Center) were prepared. 100 mL of an α-MEM medium (available from Hyclone) containing 10% fetal bovine serum (FBS, available from Gibco) and 10 ng/mL of bFGF (available from CHAmeditech) was added to a 250 mL-scale glass spinner flask (available from Corning). Each type of microbead was added thereto at a concentration of 0.02 g/mL. 2.1×10⁶ of AD-MSCs, i.e., 3,000 AD-MSCs per unit area (1 cm²), were inoculated to the microbead. Subsequently, the cells were allowed to stand for 2 hours at a temperature of 37° C. to allow cells to bind well to surfaces of the microbeads. Then, the cells were subjected to cycles in which they were allowed to stand for 10 minutes and impelled for 1 minute using an impeller such that the microbeads and cells were well adhered and dispersed. Here, the cells were cultured at 35 rpm. The culture was performed for 6 days. On the 4th day, the whole medium was replaced once.

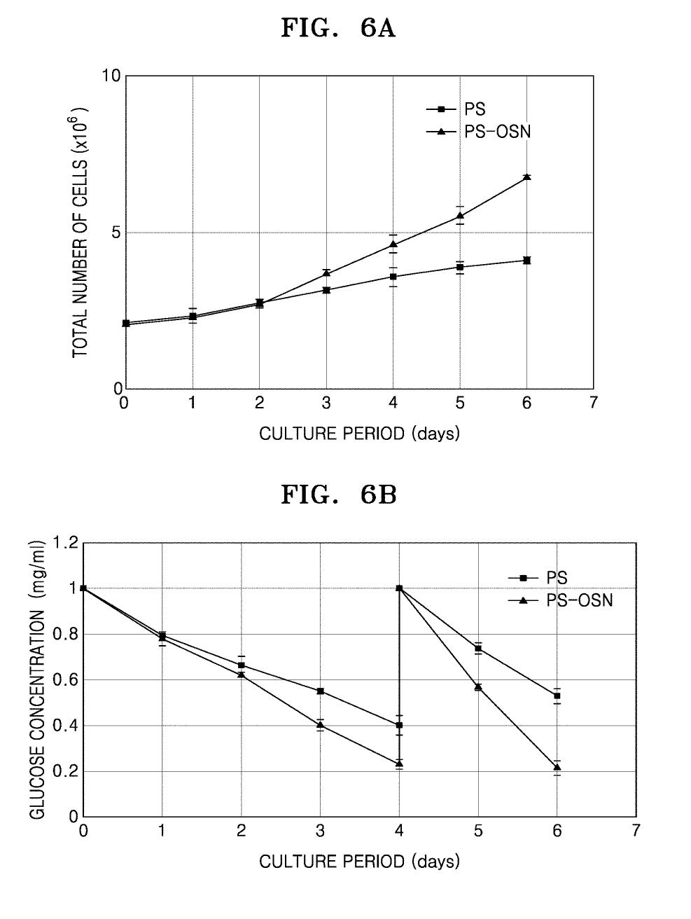

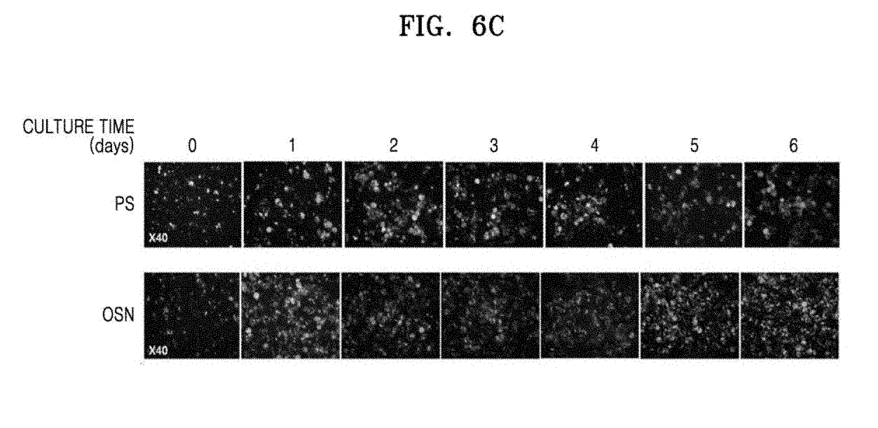

During the cell culture, 1 mL of cell culture solution was recovered every day and centrifuged. The supernatant medium was removed therefrom, and to the precipitated cells, the same amount of physiological saline solution was added for washing 2 times. The same amount of trypsin-EDTA was added, followed by reaction at a temperature of 37° C. for 5 minutes. Then, the microbeads were removed using a strainer filter having a pore size of 20 μm, and the cell suspension was recovered. The recovered cell suspension was centrifuged at 1,500 rpm for 5 minutes, the resulting cell precipitate was resuspended in 1 mL of physiological saline solution, and the number of cells was measured using a hemocytometer. FIG. 6A shows the total cell number of AD-MSCs proliferated while adhered to the microbeads during the cell culture period. Referring to FIG. 6A, with the PS microbeads, the cells proliferated to an amount of 4.12×10⁶ cells/mL±0.1 in the 6th day. With the PS-OSN microbeads, the cells proliferated to an amount of 6.77×10⁶ cells/mL±0.05 in the 6th day of culture. After 6 days of cell culture, the PS-OSN microbeads showed about a 64.5% higher cell proliferation rate than the PS microbeads.

1 mL of the cell culture solution was recovered every day during the culture period, and the concentration of glucose in the culture medium was measured immediately after the recovery using a glucose concentration assay kit (AM201 available from Asan pharmaceutical). The concentration (mg/mL) of glucose in the medium during the cell culture period is shown in FIG. 6B. Referring to FIG. 6B, by the 4th day of culture, the slope of glucose consumption with the PS microbeads was −0.15, whereas the slope with the PS-OSN microbeads was −0.19, which showed about 29% higher glucose consumption than the PS microbeads. With the PS-OSN microbeads, which showed relatively high consumption, about 29% or greater of proliferated cells were observed. The slope of glucose consumption from the 4th day to the 6th day of culture for the PS microbeads was −0.24, whereas the slope was for the PS-OSN microbeads was −0.39, which corresponds to about 67% or greater glucose consumption than the PS microbeads. The proliferation of AD-MSCs in the PS-OSN microbeads, which showed relatively high consumption during the 4th to 6th day of culture, was about four times higher during this period.

1 mL of the cell culture solution was recovered every day during the culture period, and 1 μL of 1,000 mg/mL of calcein (Sigma-Aldrich 17783-AM, in the physiological saline) was added to the recovered cell culture solution. Then, a reaction was performed at a temperature of 37° C. for 10 minutes, followed by observation using a fluorescence microscope. The cell images (×40) are shown in FIG. 6C (upper side: Col-PS bead, lower side: Col-PS-OSN bead). Referring to FIG. 6C, cells were observed to be adhered to the PS-OSN microbeads, whereas some cells as single cells were observed to be not adhered to the PS microbeads.

Regarding the culture of AD-MSCs, when PS-OSN was used, the results were the same as that of collagen-PS in the related art, and accordingly, it was found that PS-OSN is not cytotoxic to AD-MSCs. Therefore, PS—OSN may be applied to the development of a mass culture process of adult mesenchymal stem cells by using the OSN having improved abilities to load substances such as growth factors.

As apparent from the foregoing description, when a support for culturing cells, a method of preparing the support, and a method of culturing cells using the support according to one or more embodiments are used, an adherence rate, a surface area, and a proliferation rate of cells may improve, and detachment of the cells may be facilitated, thereby increasing a cell recovery rate. Also, a physiologically active substance may be slowly released, thereby reducing the cost of a cell culture process.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the disclosure as defined by the following claims.

What is claimed is:

1. A support for culturing cells, the support comprising: silica nanoparticles each having an open stoma, wherein physiologically active substance is comprised in an inner portion of the open stoma; and
a solid support to which the silica nanoparticles are bound.

2. The support of claim 1, wherein the physiologically active substance is selected from a basic fibroblast growth factor (bFGF), a brain-derived neurotrophic factor (BDNF), a vascular endothelial cell growth factor (VEGF), interleukin (IL)-4, IL-6, IL-2, an epidermal growth factor (EGF), a stem cell factor (SCF), an interferon gamma (IFNγ), and a granulocyte macrophage colony-stimulating factor (GM-CSF).

3. The support of claim 1, wherein the inner portion of the open stoma of each of the silica nanoparticles is modified with a positively charged functional group, a negatively charged functional group, or a hydrophobic functional group, and the physiologically active substance is bound to the functional group.

4. The support of claim 1, wherein the physiologically active substance is slowly released from the silica nanoparticles.

5. The support of claim 1, wherein the silica nanoparticles are based on cetylpyridinium bromide (CPB), cetyltrimethylammonium bromide (CTAB), or a combination thereof.

6. The support of claim 1, wherein the silica nanoparticles are bound to the solid support via hydrophobic interaction.

7. The support of claim 1, wherein the solid support is a bead.

8. The support of claim 7, wherein the bead is a polystyrene bead.

9. The support of claim 1, wherein the solid support is coated with collagen, fibrin, fibronectin, vitronectin, matrigel, gelatin, laminin, heparin, poly-lysine, extracellular matrix, or a combination thereof.

10. The support of claim 1, wherein a density of the silica nanoparticles on the solid support is in a range of about 1,000 per square millimeter (mm²) to about 500,000/mm².

11. The support of claim 1, wherein the support for culturing cells is sterilized.

12. A method of preparing a support for culturing cells, the method comprising:
performing, in a pressure vessel, hydrothermal treatment on a solution comprising a silica precursor, a polar solvent, a nonpolar solvent, an ionic surfactant, and a basic compound to prepare silica nanoparticles;
incubating the silica nanoparticles and a physiologically active substance for binding the physiologically active substance to an inner portion of an open stoma of each of the silica nanoparticles; and
binding, to a solid support, the silica nanoparticles to which the physiologically active substance is bound.

13. The method of claim 12, the method further comprising sterilizing the solid support to which the silica nanoparticles are bound.

14. The method of claim 12, wherein the silica precursor is tetramethyl orthosilicate, tetraethyl orthosilicate, tetrapropyl orthosilicate, or a mixture thereof; the polar solvent is water, alcohol, or a mixture thereof; and the nonpolar solvent is hexane, cyclohexane, methylcyclohexane, toluene, heptane, octane, or a mixture thereof.

15. The method of claim 12, wherein the ionic surfactant is cetylpyridinium chloride, cetylpyridinium bromide, cetyltrimethylammonium chloride, cetyltrimethylammonium bromide, or a mixture thereof; and the basic compound is an ammonia aqueous solution, urea, or a mixture thereof.

16. A method of culturing cells, the method comprising culturing cells in the presence of the support for culturing cells of claim 1.
